(19) United States
(12) Patent Application Publication
(10) Pub. No.: US 2021/0030714 A1
(43) Pub. Date: Feb. 4, 2021

(54) COMPOUNDS FOR USE IN THE TREATMENT OF BRAIN DISEASES

(71) Applicants: UNIVERSITA DEGLI STUDI DI ROMA "LA SAPIENZA", Roma (RM) (IT); FONDAZIONE ISTITUTO ITALIANO DI TECNOLOGIA, Genova (GE) (IT); UNIVERSITA DEGLI STUDI DI PADOVA, Padova (IT)

(72) Inventors: Cristina LIMATOLA, Roma (IT); Giuseppina D'ALESSANDRO, Roma (IT); Lucia DI MARCOTULLIO, Roma (IT); Paola INFANTE, Genova (IT); Bruno BOTTA, Roma (IT); Mattia MORI, Genova (IT); Francesca GHIRGA, Genova (IT); Cinzia INGALLINA, Roma (IT); Simone BERARDOZZI, Roma (IT); Paolo CALICETI, Padova (IT); Stefano SALMASO, Padova (IT); Michela DE MARTINO, Roma (IT); Francesco GASPARRINI, Roma (IT); Deborah QUAGLIO, Roma (IT)

(73) Assignees: UNIVERSITA DEGLI STUDI DI ROMA "LA SAPIENZA", Roma (RM) (IT); FONDAZIONE ISTITUTO ITALIANO DI TECNOLOGIA, Genova (GE) (IT); UNIVERSITA DEGLIS STUDI DI PADOVA, Padova (IT)

(21) Appl. No.: 16/967,006

(22) PCT Filed: Feb. 5, 2019
(86) PCT No.: PCT/EP2019/052800

§ 371 Date: Aug. 3, 2020

(30) Foreign Application Priority Data
Feb. 5, 2018 (IT) 102018000002402

(51) Int. Cl. A61K 31/366; A61K 47/40; A61P 35/04

(52) U.S. Cl. CPC A61K 31/366; A61K 9/0043; A61P 35/04; A61K 47/40

(57) ABSTRACT

The present invention relates to a compound of formula (1):

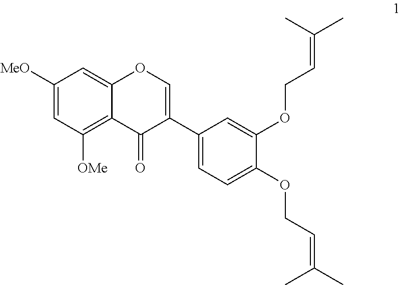 [SMILES: COc1cc(OC)c2c(=O)c(-c3ccc(OCC=C(C)C)c(OCC=C(C)C)c3)coc2c1]

or an analogue thereof or a pharmaceutically acceptable salt thereof, for use in the treatment of a brain tumor, wherein the compound is administered intranasally and relative pharmaceutical compositions.

### TECHNICAL FIELD OF THE INVENTION

The present invention relates to a method for rapidly and efficiently distributing a compound of formula (1)

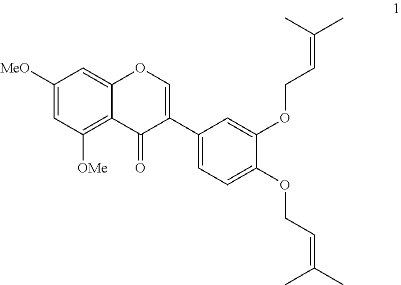 [SMILES: COc1cc(OC)c2c(=O)c(-c3ccc(OCC=C(C)C)c(OCC=C(C)C)c3)coc2c1]

or analogous compounds thereof and/or pharmaceutically acceptable salts thereof, alone or in conjunction with other compounds and relative pharmaceutical compositions. In particular the compounds reach the systemic circulation through nasal administration. Then a rapid onset of beneficial effects for the treatment and/or the prevention of brain tumors is achieved.

### BACKGROUND ART

Glioblastoma is the most widespread and aggressive neoplasm of the central nervous system (CNS), characterized by a high level of invasion and proliferation of tumor cells and by a high level of inflammation and necrosis of the nervous tissue (1). Despite the ongoing progress of neurosurgery and in the formulation of new medicaments, at present there are no effective therapies to counter the growth of this brain tumor and patient's death occurs within about twelve months from diagnosis of the pathology (2).

A cause which often hinders the effectiveness of a pharmacological treatment for brain tumors is the achievement and maintenance of therapeutic concentrations of the medicament in the brain to be treated. This organ is highly protected against external agents (chemical or biological) by the blood-brain barrier. However, this defensive mechanism thwarts therapeutic interventions: about 98% of medicaments do not cross the blood-brain barrier (3).

Recently, clinical studies on intranasal administration of medicaments for targeting brain tumors and, specifically, glioblastoma are significantly increasing. Considering that the brain and the nasal cavity are directly connected to each other through the olfactory and trigeminal pathway, the obstacle of the blood-brain barrier can be overcome by intranasal administration (4). Furthermore, the intranasal administration involves the possibility of reaching therapeutic concentrations using lower amounts of medicament, therefore avoiding possible peripheral side effects (4).

Therefore, there is the need to find a method of administration of a medicament which allows to overcome the blood-brain barrier and to maintain therapeutic concentrations of the medicament in the brain for the treatment of tumors of the central nervous system, in particular of brain tumors, more particularly Hedgehog-dependent tumors, preferably glioma or glioblastoma.

Another cause which often makes experimental treatments ineffective for glioblastoma is its multiform nature. Primary glioblastoma presents in most cases a normal p53 oncosuppressive function (5), but at the same time the expression of staminality markers such as nestin, Notch3 and the Sonic Hedgehog (SHH) ligand in a subpopulation of malignant cells, called cancer stem cells is observed (6, 7). Glioblastoma cells expressing staminality markers are resistant to a very high number of medicaments and radiations and show a continuous activation of the Hedgehog (Hh) signalling pathway (8).

Therefore, there is a need to find a treatment which is effective against glioblastoma, in particular against the cells expressing staminality markers such as nestin, Notch3 and the Sonic Hedgehog (SHH) ligand.

The activity of the Hedgehog signalling pathway is due to the binding of the Hh ligands (i.e. Sonic Shh, Indian Ihh and Desert Dhh) to the PTCH (Patched) membrane receptor. This interaction reduces the inhibitory activity of PTCH on the SMO (Smoothened) transducer, a receptor with 7 transmembrane domains. In turn, SMO activates downstream transcription factors belonging to the Gli (Gli1, Gli2 and Gli3) family, acting on a series of target genes promoting cell proliferation and reducing cell differentiation. These target genes include the Gli1 itself, thus strengthening the activation of the Hedgehog signalling pathway. Gli1 is the key effector of the Hedgehog signalling pathway. in the fact the mRNA levels of the transcription factor itself are considered a significant indication of signalling pathway activity in tumors (9). Recently it has been shown that on a large cohort (n=149) of tissues from patients suffering from glioblastoma multiforme (GBM), the Hedgehog pathway is active, supported by constant mRNA levels of Gli1 in all analysed samples (10).

Glabrescione B (GlaB) is an organic compound of formula (1), an inhibitor of the Sonic Hedgehog pathway in different tumor models (medulloblastoma, basal cell carcinoma) (11, 12).

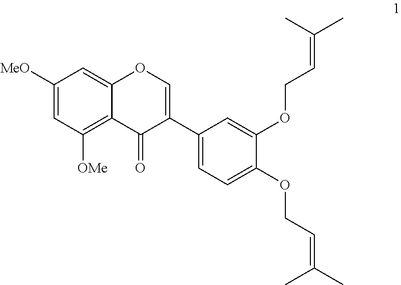 [SMILES: COc1cc(OC)c2c(=O)c(-c3ccc(OCC=C(C)C)c(OCC=C(C)C)c3)coc2c1]

GlaB is an isoflavone having the chemical formula (1), which is naturally present in the seeds of Derris Glabrescens (Leguminosae). Its formula comprises the core of 5,7-dimethoxyisoflavone and can be obtained according to Delle Monache, F.; et al. (1977), Gazzetta Chimica Italiana 107(7-8): 403-407. International patent application WO 2014/207069 discloses GlaB and a series of analogues thereof as selective inhibitors of the activity of the Hedgehog signalling pathway (Hh), preparation methods and uses thereof. GlaB acts by countering the interaction between the transcriptional factor Gli1 and DNA and therefore inhibits the transcriptional activity of factors belonging to the family of Gli proteins.

### SUMMARY OF THE INVENTION

In the present invention it was surprisingly found that intranasal administration of GlaB is extremely advantageous. In fact, intranasal administration allows GlaB to significantly penetrate the blood-brain barrier and reach the brain in therapeutically significant amounts. Further, the amount of GlaB necessary to treat brain tumors is significantly lower when GlaB is administered intranasally compared to other routes. Moreover, thanks to the intranasal administration, GlaB is confined to the brain and does not elicit side effects.

The present invention provides a safe and convenient method for administering a compound of formula (1) or analogous compounds thereof or pharmaceutically acceptable salts thereof to a subject (mammal or human) to prevent and/or treat a brain tumor. The therapeutic effect is achieved quickly and effectively. The method comprises the administration of a pharmaceutically acceptable amount of a compound of formula (1) or a composition comprising it to the brain of a subject suffering from or at risk of developing a brain tumor, wherein the administration to the brain includes intranasal administration of the compound or composition. Preferably, the composition is administered directly to the nasal epithelium of the subject or into the upper nasal cavity, so as to overcome the blood-brain barrier and deliver the therapeutic composition directly to the central nervous system. The present invention is further advantageous in that it improves the rate of administration of a compound of formula (1) or analogous compounds thereof or pharmaceutically acceptable salts thereof in the systemic circulation by administrating nasally a compound of formula (1) or analogous compounds thereof or pharmaceutically acceptable salts thereof in order to accelerate the onset of therapeutic effects and/or to reduce the dose necessary to obtain beneficial effects.

The intranasal administration improves the bioavailability of the medicament by direct absorption into the blood, thus avoiding a large first-pass metabolism which can significantly reduce plasma concentrations of a compound of formula (1) or analogous compounds thereof or pharmaceutically acceptable salts thereof when they are administered to other pathways. Accordingly, small doses of a compound of formula (1) or analogous compounds thereof or pharmaceutically acceptable salts thereof may be administered, resulting in less side effects and greater tolerability and efficacy in subjects suffering from a brain tumor. Moreover, since a compound of formula (1) or analogous compounds thereof or pharmaceutically acceptable salts thereof are rapidly effective after the intranasal administration, the selection of an ideal dose for a particular subject is greatly facilitated. In the present invention, intranasal dosage forms containing a compound of formula (1) or analogous compounds thereof or pharmaceutically acceptable salts thereof in combination with other medicaments used in the treatment of brain tumors may also be used.

Intranasal administration is particularly easy to practice since relatively simple devices have already been mass-produced to this purpose. A compound of formula (1), an analogue thereof, pharmaceutically acceptable salts thereof and the pharmaceutical composition of the invention are therefore preferably adapted for and/or packaged for intranasal administration, for example, as a nasal spray, nasal drops, aerosol, nasal gel or nasal powder.

With the aforementioned and further objectives, advantages and characteristics of the invention which will become apparent in the following, the nature of the invention is further explained in the following detailed description of the preferred embodiments of the invention and in the appended claims.

Therefore, it is an object of the present invention a compound of formula (1):

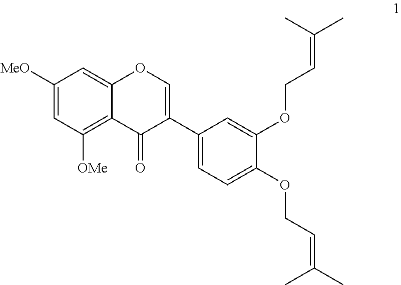 [SMILES: COc1cc(OC)c2c(=O)c(-c3ccc(OCC=C(C)C)c(OCC=C(C)C)c3)coc2c1]

or an analogue thereof or a pharmaceutically acceptable salt thereof, for use in the treatment of a brain tumor, wherein said compound is administered intranasally. Preferably, the brain tumor is a glioma. More preferably, the tumor is a glioblastoma.

In a preferred aspect of the invention, the brain tumor is dependent on the Hedgehog (Hh) signalling pathway. In a further preferred aspect, the compound of general formula (1) or analogue thereof or pharmaceutically acceptable salt thereof acts as an antagonist of Gli1 and/or of SMO.

Preferably, the brain tumor is characterized by the presence of cells expressing at least one marker selected from the group consisting of: nestin, Notch3 and the Sonic Hedgehog (SHH) ligand.

Still preferably, the brain tumor is a primary tumor or a metastasis. Even more preferably, the brain tumor is resistant to at least one medicament and/or to radiations.

In a further preferred aspect of the invention, the compound or analogue thereof or pharmaceutically acceptable salt thereof is administered every two days.

Preferably, the compound or analogue thereof or pharmaceutically acceptable salt thereof is administered for at least 6 times. More preferably, the compound or analogue thereof or pharmaceutically acceptable salt thereof is administered every two days for 6 times.

Preferably, the compound or analogue thereof or pharmaceutically acceptable salt thereof is administered for at least a 6-day cycle. More preferably, the compound or analogue thereof or pharmaceutically acceptable salt thereof is administered every two days for at least a 6-day cycle.

Preferably, the compound or analogue thereof or pharmaceutically acceptable salt thereof is administered at a concentration in a range from about 1 to about 10 mg/kg, preferably at a concentration of about 4.4 mg/kg or at a concentration of about 1.4 mg/Kg.

In a preferred embodiment, the compound or an analogue thereof or the pharmaceutically acceptable salt thereof is administered every two days, for at least 6 times, preferably at a concentration comprised from approximately 1 to approximately 10 mg/Kg, preferably at a concentration of approximately 4.4 mg/Kg or at a concentration of approximately 1.4 mg/Kg.

A further object of the present invention is the compound (1) or analogue thereof or pharmaceutically acceptable salt thereof for use according to the invention in conjunction with at least one further therapeutic intervention. Preferably, the further therapeutic intervention is a surgical operation, a radiation therapy or a treatment with a further therapeutic agent. Preferably, said further therapeutic agent is an alkylating agent or an anti-angiogenic agent.

In a further aspect, the present invention provides a pharmaceutical composition comprising a compound of formula (1) or an analogue thereof or a pharmaceutically acceptable salt thereof and a pharmaceutically acceptable excipient and/or diluent for use in the treatment of a brain tumor, wherein said composition is administered intranasally.

Preferably the pharmaceutically acceptable excipient and/or diluent is selected from the group consisting of: a hydrophilic polymer, a hydrophobic molecule, an alcohol, a cyclodextrin, a polyoxyl hydrogenated castor oil, a polyoxyl castor oil, water and a mixture or conjugate thereof.

Preferably, the hydrophilic polymer is PEG. Preferably, the hydrophobic molecule is cholane, cholesterol, a phospholipid, or an alkyl chain. Preferably, the alcohol is ethanol.

Preferably, the cyclodextrin is an α-, β-, or γ-cyclodextrin, a semisynthetic cyclodextrin (such as a methyl cyclodextrin with different degrees of substitution of hydroxyl groups), or another pharmaceutically acceptable cyclodextrin. Also preferably, the cyclodextrin is 2-hydroxypropyl-beta-cyclodextrin. Preferably, the polyoxyl hydrogenated castor oil is polyoxyl 40 hydrogenated castor oil or polyoxyl 60 hydrogenated castor oil. Preferably, the polyoxyl castor oil is polyoxyl 35 castor oil.

Still preferably the pharmaceutically acceptable excipient and/or diluent is a conjugate of PEG and Cholane or a mixture of ethanol and 2-hydroxypropyl-beta-cyclodextrin.

Preferably, the 2-hydroxypropyl-beta-cyclodextrin is in the form of a solution.

Preferably, PEG and Cholane are in a 1:1 molar ratio.

Preferably, the 2-hydroxypropyl-beta-cyclodextrin solution is in a concentration of 10% w/v in water. Also preferably, ethanol and the solution are in a 1:5 volume ratio. Then preferably, the excipient and/or diluent is a mixture of ethanol and 2-hydroxypropyl-beta-cyclodextrin solution (10% (w/v) in water) in a 1:5 ratio (vol/vol).

Preferably, the pharmaceutical composition comprises at least one further therapeutic agent.

In a further aspect, the present invention provides a pharmaceutical composition comprising a compound of formula (1) or an analogue thereof or a pharmaceutically acceptable salt thereof and a pharmaceutically acceptable excipient and/or diluent, wherein said excipient and/or diluent is selected from the group consisting of: a hydrophilic polymer, a hydrophobic molecule, a polyoxyl hydrogenated castor oil, a polyoxyl castor oil, and a mixture or conjugate thereof. Said pharmaceutical composition optionally comprises at least one further therapeutic agent. Preferably, the hydrophilic polymer is PEG. Preferably, the hydrophobic molecule is cholane, cholesterol, a phospholipid, or an alkyl chain. Preferably, the polyoxyl hydrogenated castor oil is polyoxyl 40 hydrogenated castor oil or polyoxyl 60 hydrogenated castor oil. Preferably, the polyoxyl castor oil is polyoxyl 35 castor oil.

Preferably, the pharmaceutically acceptable excipient and/or diluent is a conjugate of PEG and Cholane. Preferably, PEG and Cholane are in a 1:1 molar ratio.

Preferably, the further therapeutic agent is an alkylating agent or an anti-angiogenic agent.

Preferably, the further therapeutic agent is a ligand of a growth factor or a receptor thereof, e.g. VEGF, TGF-β2, pan-VEGFR, VEGFR2, VEGFR, PDGFR-β.

Preferably, the further therapeutic agent is a ligand of an intracellular effector, for example: PKC-β, PI3K/Akt, mTOR, bcl-2, RAF, RAS, PARP-1.

Preferably, the further therapeutic agent is a kinase inhibitor.

Preferably, the further therapeutic agent is selected from the group consisting of: temozolomide, doxorubicin, bevacizumab, irinotecan, fotemustine, fosbrezitabulin, trabedersen, cediranib, vatalanib, sorafenib, sunitinib, tandutinib, lomustine, vincristine, apatinib, everolimus, dasitinib, topotecan, nivolumab, nelfinavir, vandetanib, crenolanib, cilengitide, rapamycin, lenvatinib, carmustine, naltrexone, enzastaurin, carboplatin, capecitabine and nimotuzumab.

In the present invention, the expressions “analogous compound(s) of the compound of formula (1)” and, more simply, “analogue of a compound of formula (1)” refer to compounds structurally or functionally similar to the compound of formula (1). For example, an analogue of a compound of formula (1) may have a different chemical structure compared to the compound of formula (1), maintaining the pharmacophoric characteristics.

WO 2014/207069 discloses GlaB and a series of analogues thereof as selective inhibitors of the activity of the Hedgehog signalling pathway (Hh), preparation methods and uses thereof.

WO 2014/207069 is incorporated by reference. Salmaso, S. et al. Self-assembling nanocomposites for protein delivery: Supramolecular interactions between PEG-cholane and rh-G-CSF. Journal of Controlled Release 162, 176-184, (2012) and Ambrosio, E. et al. A novel combined strategy for the physical PEGylation of polypeptides. Journal of Controlled Release 226, 35-46 (2016) are incorporated by reference.

Examples of analogue compounds of the compound of formula (1) are described in WO/2014/207069 and include:

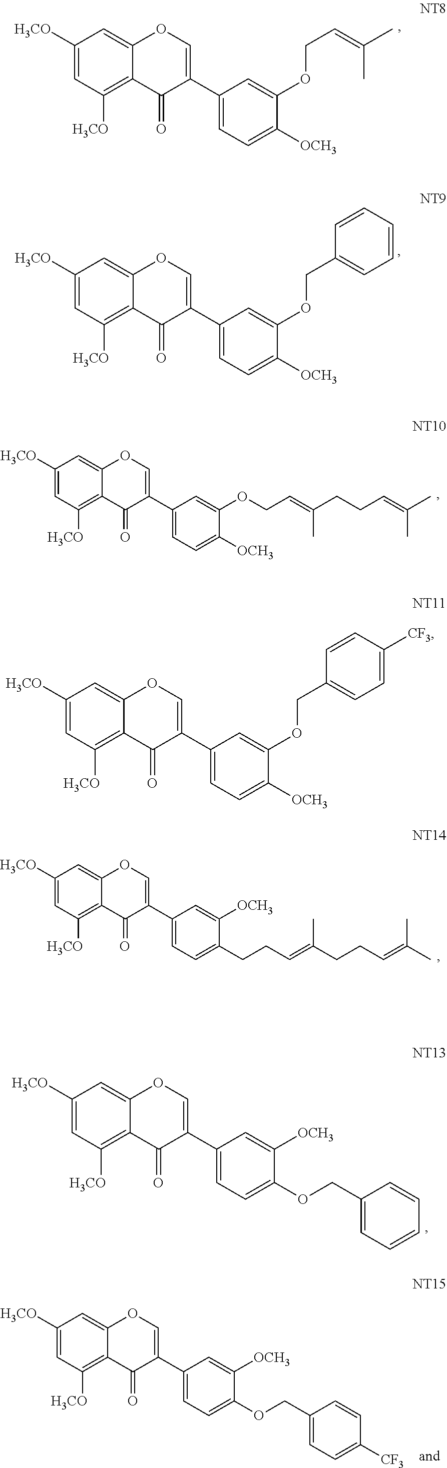

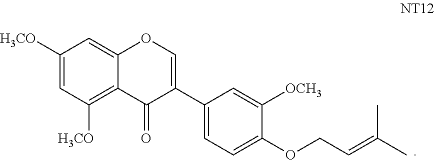 [SMILES: COc1cc(OC)c2c(=O)c(-c3ccc(OCC=C(C)C)c(OC)c3)coc2c1]

In the present invention, the compound of formula (1) or an analogue thereof for use in the treatment of a brain tumor by intranasal administration may be in the form of a pharmaceutically acceptable salt. Pharmaceutically acceptable salts conventionally include non-toxic salts obtained by salification of a compound of formula (1) or an analogue thereof with inorganic acids (e.g. hydrochloric, hydrobromic, sulfuric or phosphoric acid), or with organic acids (e.g. acetic, propionic, succinic, benzoic, sulfanilic, 2-acetoxy-benzoic, cinnamic, mandelic, salicylic, glycolic, lactic, oxalic, malic, maleic, malonic, fumaric, tartaric, citric, p-toluenesulfonic, methanesulfonic, ethanesulfonic acid, or naphthalenesulfonic acids). For information on pharmaceutically suitable salts, see Berge S. M. et al., J. Pharm. Sci. 1977, 66, 1-19; Gould P. L. Int. J. Pharm 1986, 33, 201-217; and Bighley et al. Encyclopedia of Pharmaceutical Technology, Marcel Dekker Inc, New York 1996, Volume 13, pages 453-497.

In addition, pharmaceutically acceptable salts obtained by addition of a base can be formed with a suitable inorganic or organic base such as triethylamine, ethanolamine, triethanolamine, dicyclohexylamine, ammonium hydroxide, pyridine. The term “inorganic base”, as used herein, has its ordinary meaning as understood by one of ordinary skill in the art, and generally refers to an inorganic compound which can act as a proton acceptor. The term “organic base”, as used herein, also has its ordinary meaning as understood by one of ordinary skill in the art and generally refers to an organic compound that can act as a proton acceptor.

Other suitable pharmaceutically acceptable salts include pharmaceutically acceptable alkaline metals or alkaline earth metals such as sodium, potassium, calcium or magnesium salts; in particular, pharmaceutically acceptable salts of one or more portions of carboxylic acids which may be present in the compound of formula (1) or an analogue thereof. Furthermore, the compound of formula (1) or an analogue thereof can be administered in non-solvated forms as well as in solvated forms with pharmaceutically acceptable solvents such as water, EtOH and the like.

The compound of formula (1) or an analogue thereof may exist in stereoisomeric forms (for example, they may contain one or more asymmetric carbon atoms). The individual stereoisomers (enantiomers and diastereomers) and mixtures thereof can be administered intranasally according to the present invention. The present invention covers the individual isomers of the compound of formula (1) or an analogue thereof as well as mixtures with isomers in which one or more chiral centres are inverted for use in the treatment of a brain tumor by intranasal administration.

Similarly, it is understood that the compound of formula (1) or an analogue thereof may exist in tautomeric forms different from those shown in the formula and these for use according to the present invention are included within the scope of the present invention. The invention also includes all the isotopic variants of a compound of the invention for use in the treatment of a brain tumor by intranasal administration. An isotopic variant of a compound of the invention is defined as a variant in which at least one atom of the molecule is replaced by an atom having the same atomic number but an atomic mass which is different from the atomic mass usually present in nature. Examples of isotopes that may be incorporated into the compounds of the invention include isotopes such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variants of the invention, for example, those in which a radioactive isotope such as ³H or ¹⁴C is incorporated, are used in studies of tissue distribution of medicaments and/or substrates. Furthermore, replacement with isotopes such as ²H deuterium can lead to therapeutic benefits resulting from increased metabolic stability. Isotopic variants of the compounds of the invention can generally be prepared by conventional procedures using suitable isotopic variants of suitable reagents.

The compounds of the invention can be conveniently administered nasally by formulating them in a nasal pharmaceutical composition comprising a compound of formula (1) and/or an analogue thereof and a non-toxic pharmaceutically acceptable nasal vehicle. In the pharmaceutical composition, the compound of formula (1) and/or the analogue thereof may be used as a free base or as a pharmaceutically acceptable salt thereof, as detailed above. Non-toxic, non-irritating, pharmaceutically acceptable nasal vehicles will be apparent to those skilled in the art of nasal pharmaceutical formulations. Examples of pharmaceutically acceptable nasal vehicles include: water; physiological saline; alcohols such as ethanol and isopropanol; glycols such as propylene glycol; glycol ethers, such as polyethylene glycols which are ethylene oxide and water polymers, represented by the formula H(OCH₂—CH₂)ₙOH, wherein n ranges from 5 to 10.

Other ingredients may also be present, such as: buffers, preservatives, osmotic agents, gelling agents, wetting agents. Examples of buffers which may be used in the compositions of this invention are: glycine; citric acid and alkaline salts thereof; acetic acid and alkaline salts thereof; phosphoric acid and alkaline salts thereof; gluconic acid and alkaline salts thereof; sodium hydroxide and potassium hydroxide. The preservatives useful in the compositions include: benzalkonium chloride, cetalkonium chloride, cetyl pyridinium chloride, cetyl trimethyl ammonium bromide, chlorobutanol, methylparaben, propylparaben, phenyl mercuric acetate, thiomer and the like. Examples of osmotic agents include sorbitol, sodium chloride and the like. Examples of gelling agents include methylcellulose, sodium carboxymethylcellulose, hydroxypropyl cellulose, hydroxypropylmethylcellulose, hydroxyethyl cellulose, xanthan gum and the like. Useful wetting agents include polysorbate 60 or 80 and other fatty esters and polyethylene glycol ethers, quaternary ammonium salts, alkylphenoxy polyethylene glycols, polyethylene block polymers and polypropylene oxides and the like.

The compositions may also include one or more mucosal adjuvants known to those skilled in the art.

A reference for the formulation is the book by Remington (“Remington: The Science and Practice of Pharmacy”, Lippincott Williams & Wilkins, 2000).

The composition of the invention is suitable to be administered intranasally, being for example in the form of a nasal solution; a nasal suspension; a nasal ointment; a nasal gel; a nasal cream; an inhalation preparation. In particular, the nasal solution can be in the form of: drops, sprays or aerosols. The inhalation preparation can be administered with a pressurized insufflator or nebulizer.

When administering a nasal dosage form such as spray or aerosol, a propellant gas can be added to the active ingredient and the vehicle composition. Suitable propellant gases include polyhalogenated alkanes, such as trichloromonofluoromethane, CCl₃F (Freon 11); dichlorodifluoromethane, CCl₂F₂ (Freon 12); 1,2-dichloro-1,1,2,2-tetrafluoroethane, CClF₂ (Freon 114) and mixtures thereof. The nasal formulation as a spray or aerosol can also be administered by mechanical devices without the use of propellant gases.

In a preferred embodiment, the compound of formula (1) or an analogue thereof or pharmaceutically acceptable salts thereof for use according to the present invention is formulated in polymer nanocapsules as described in C. Ingallina, P. M. Costa, F. Ghirga, R. Klippstein, J. T. Wang, S. Berardozzi, N. Hodgins, P. Infante, S. M. Pollard, B. Botta, K. T. Al-Jamal. Polymeric glabrescione B nanocapsules for passive targeting of Hedgehog-dependent tumor therapy in vitro. Nanomedicine, (2017), 12(7), 711-728. DOI: 10.2217/nnm-2016-0388.

Formulations of the compound of the invention/PEG-Cholane can be obtained with PEG-Cholane as excipient in aqueous media to promote dissolution and/or stabilization of the dispersed compound of the invention. PEG-Cholane is used because its self-assembling properties yield colloidal systems that encapsulate the compound or as surface coating agent of dispersions of the compound. Formulations can be obtained with different PEG-Cholane concentrations (0-100 mg/mL).

PEG-Cholane include a family of conjugates of the polymeric hydrophilic material Polyethylene glycol (PEG) and Cholane; Cholane is intended to be chemically conjugated to one terminal end of PEG chain through an amide, ester, urethane, or ether bond with or without chemical spacers. PEG can have different molecular weight (500-50,000 Da, preferably 5000 Da, also preferably PEG400) and can be linear or branched. Other medically approved hydrophilic polymers (including but not limited to polyvinyl pyrrolidone (PVP), a polyvinyl alcohol (PVA), a polyacrylic acid (PAA), copolymers of PAA modified with block-copolymers of poly(ethylene oxide) (PEO) and polypropylene oxide) PPO, a polyacrylamide, N-(2-hydroxypropyl) methacrylamide (HPMA), divinyl ether-maleic anhydride (DIVEMA), a polyphosphate (PPE), a polyphosphazene) can be used instead of PEG. Cholane can be conjugated in multiple copies to one end of PEG with suitable linkers. Other hydrophobic molecules can be used instead of Cholane including, but not limited, to Cholesterol, phospholipids and alkyl chains.

Other pharmaceutically acceptable alcohols may be used in addition to or in place of ethanol in the compositions according to the present invention.

Cyclodextrins are cyclic oligosaccharides consisting of 6, 7, or 8 glucopyranose units, usually referred to as α-, β-, or γ-cyclodextrins, respectively. These compounds have rigid doughnut-shaped structures making them natural complexing agents. The unique structures of these compounds owe their stability to intramolecular hydrogen bonding between the C2- and C3-hydroxyl groups of neighboring glucopyranose units. The molecule takes on the shape of a torus with the C2- and C3-hydroxyls located around the larger opening and the more reactive C6-hydroxyl aligned around the smaller opening. The arrangement of C6-hydroxyls opposite the hydrogen bonded C2- and C3-hydroxyls forces the oxygen bonds into close proximity within the cavity, leading to an electron rich, hydrophobic interior. The size of this hydrophobic cavity is a function of the number of glucopyranose units forming the cyclodextrin. β-cyclodextrin is most commonly used. Other cyclodextrins suitable for use in the formulations according to the invention include, but are not limited to, α-, β-, or γ-cyclodextrins, semisynthetic cyclodextrins such as, methyl cyclodextrins with different degrees of substitution of their hydroxyl groups, and other pharmaceutically approved cyclodextrins.

Alternative excipients used to prepare the formulations of the invention include emulsifying and solubilising agents, stabilizing agents of drug dispersions, and compositions of agents including, but not limited to, Polyoxyl n castor oil (n=30 to 40) (synonyms: ethoxylated castor oil, polyethylene glycol castor Oil). Polyoxyl n castor oil is a mixture of triricinoleate esters of ethoxylated glycerol with small amounts of polyethyleneglycol (macrogol) ricinoleate and the corresponding free glycols. The number (n) associated with the name of the substance represents the average number of oxyethylene units in the compound. Polyoxyl n hydrogenated castor oil (n=40 to 60) is a mixture of trihydroxystearate esters of ethoxylated glycerol with small amounts of macrogol trihydroxystearate and the corresponding free glycols. The substances are generally highly dispersible in water. Polyoxyl castor oil and polyoxyl hydrogenated castor oil are nonionic surfactants, which are used as emulsifying and solubilising agents in pharmaceutical preparations and cosmetics. Examples are polyoxyl 35 castor oil (Cremophor EL; CAS 61791-12-6), polyoxyl 40 castor oil (Marlowet 40, Emulgin RO 40), polyoxyl 40 hydrogenated castor oil (Cremophor RH 40) and polyoxyl 60 hydrogenated castor oil (Cremophor RH 60). The substances are included as excipients in numerous preparations intended for use in all food producing species by parenteral, oral or topical administration. The concentration in products is usually between 0.1% and 20% with a maximum of 27.5%. The doses of concentrated substances to different species is in a range of 0.01 and 2.5 mL/day (cattle and horses 0.75 to 2.5 ml, sheep and goats 0.2 to 0.5 ml, swine 0.25 to 1.20 ml, poultry 0.001 to 0.03 ml and salmon as a dip for 30 minutes in a 36% solution diluted ⅓×10⁶ before use). Pharmaceutical compositions for use in the treatment of a brain tumor by intranasal administration can be prepared by procedures well known to those skilled in the art.

The compounds of the present invention may be used intranasally in the treatment and/or prevention of the aforementioned conditions as single therapy or in combination with other therapeutic agents, either through separate administration or including two or more active ingredients in the same pharmaceutical formulation. The compounds may be administered simultaneously or sequentially. Furthermore, further aspects include the combination of the compounds of the invention described herein with other therapies for brain tumors for a greater synergistic benefit. The other therapeutic agents may be other medicaments approved for the treatment of brain tumors.

The combination of individual treatment compounds can be administered in a separate (simultaneous or sequential) composition or as a single dosage containing all agents.

When the compounds of the invention are combined with other active ingredients, these can be formulated separately in single principle preparations of one of the above compounds and made as a combined preparation, to be administered in equal or different times, or again formulated in combination with two or more active ingredients.

The compound of formula (1) or the analogue thereof can be administered to a patient in a total daily dose of, for example, 0.001-1000 mg/kg of body weight per day. The unit dosage compositions can contain these amounts of submultiples of the same to reach the daily dose. The compound can also be administered weekly, daily or every two days. The determination of optimal dosages for a particular patient is a process well known by those skilled in the art. A typical dose of the composition for intranasal use has a volume ranging from 0.1 μl to 100 μl, in two sprays, one per nostril.

As is common practice, the compositions are generally accompanied by written or printed instructions for use in the treatment in question.

The present invention will be described by means of non-limiting examples, referring to the following figures:

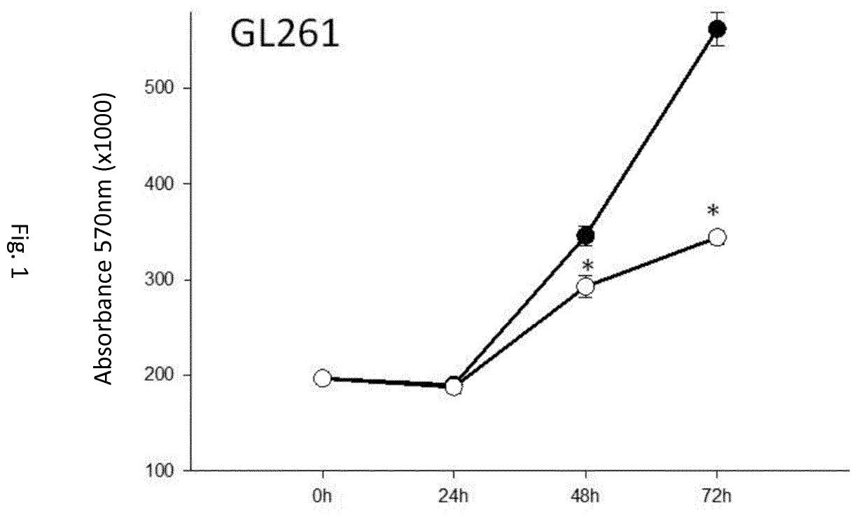

FIG. 1. GL261 cell growth curve evaluated by the MTT salt assay. Statistical analysis using ANOVA, Student-Neuman-Keuls post-test showed a significant reduction (N=3*p<0.001) of the growth of tumor cells treated with 5 μM GlaB (white circles) in vitro starting from 48 h of treatment compared to the cells treated only with the vehicle (DMSO) (black circles).

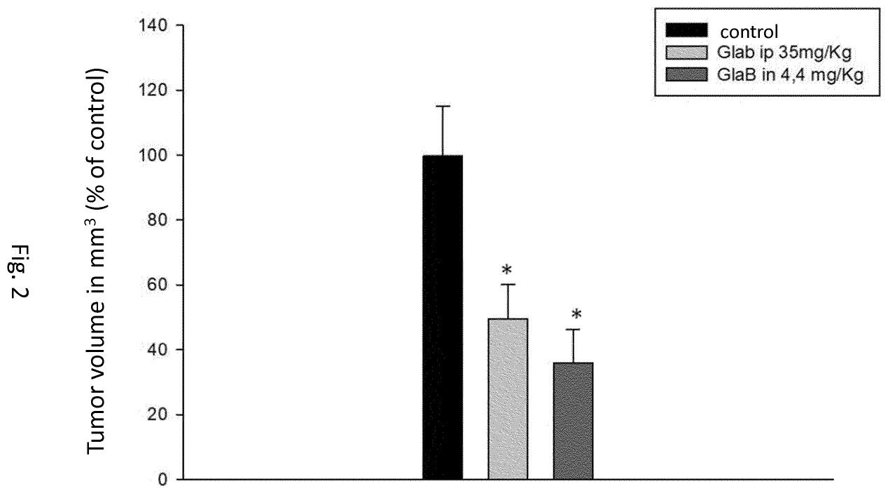

FIG. 2. Histogram of the tumor volume measured in mm³ in mice inoculated with murine glioma GL261 cells. Statistical analysis using Student's T-test showed a significant reduction (*p<0.001) of tumor volume in mice treated with intraperitoneal (N=7, 35 mg/kg), (0.48+0.10 mm³) and intranasal (N=7, 4.4 mg/kg) (0.21+0.04 mm³) GlaB compared to control mice treated intranasally (IN) with the vehicle alone (ethanol: 2-hydroxypropyl-beta-cyclodextrin aqueous solution, 1:5 v:v) (N=10, 0.96+0.18 mm³).

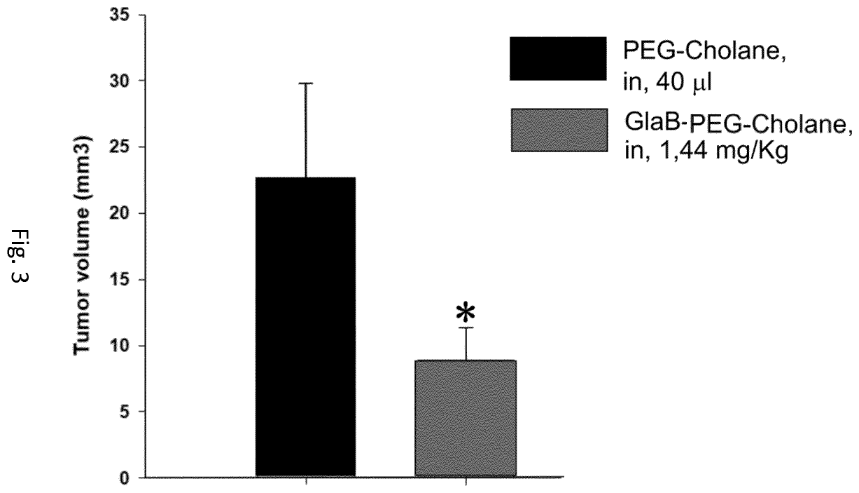

FIG. 3. Tumor volume bar graph (measured as mm³ volume) in mice injected with murine glioma cells (GL261). Student's T-test analysis revealed a significant difference (*p<0.05) between mice treated with intranasal (in) GlaB-PEG-Cholane (N=8, 1.44 mg/Kg) and mice treated only with intranasal (in) vehicle PEG-Cholane (40 μl).

FIG. 4. Chromatograms for estimated limit of quantitation (LOQ) in UV and MS: (A) LOQ in UV determination is 4.1 ng of GlaB on column with a S/N (signal to noise ratio)=13.1 for UV chromatogram and a S/N=194.9 for MS trace; (B) LOQ in MS determination is 0.26 ng of GlaB on column with a S/N=1.2 for UV chromatogram and a S/N=12.4 for MS trace.

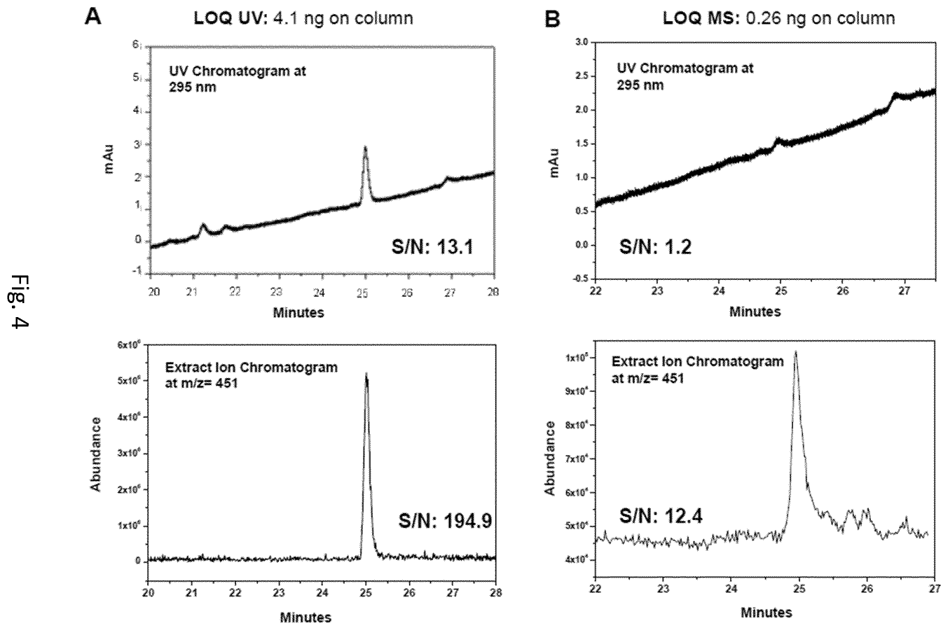

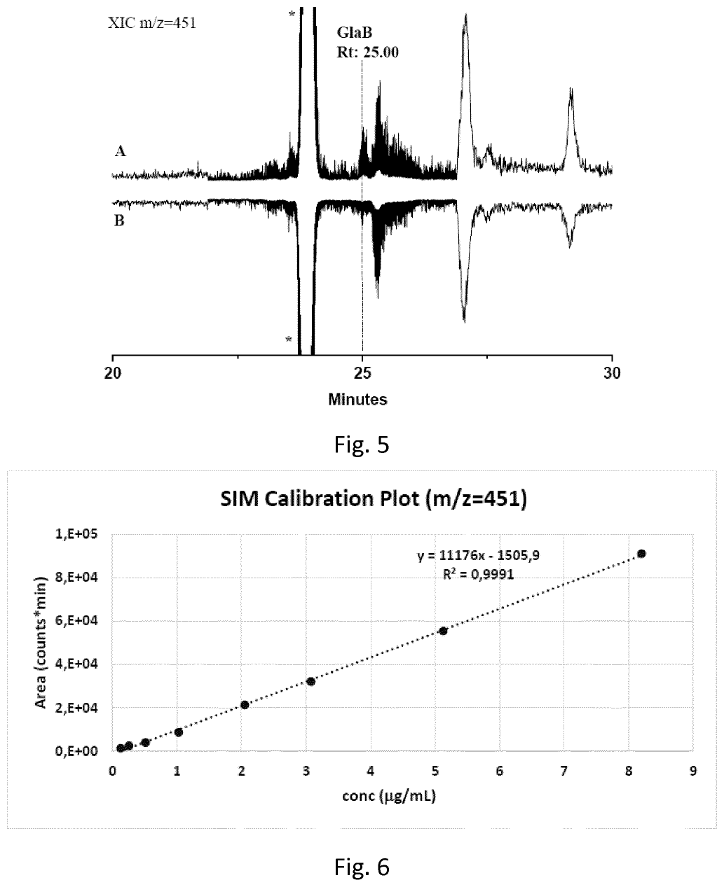

FIG. 5. Extract ion chromatograms (XIC) referred to brain extract of IN GlaB/PEG-Cholane treated mice (A) and control PEG-Cholane treated mice (B). GlaB peak (Rt, retention time: 25.00 min) is detected in IN treated sample and is missing in control. *The peak at Rt: 23.90 min (marked with an asterisk in FIG. 5) is the isotopic abundance with m/z=451 of an unknown peak with m/z=499, present in all brain extracts.

FIG. 6. GlaB quantification with HPLC-MS. Linear regression plots of different GlaB concentrations from 0.128 to 8.2 μg/mL in a mixture of methanol/water (4:1) (SIM=451). Results are expressed as means±SD (n=3).

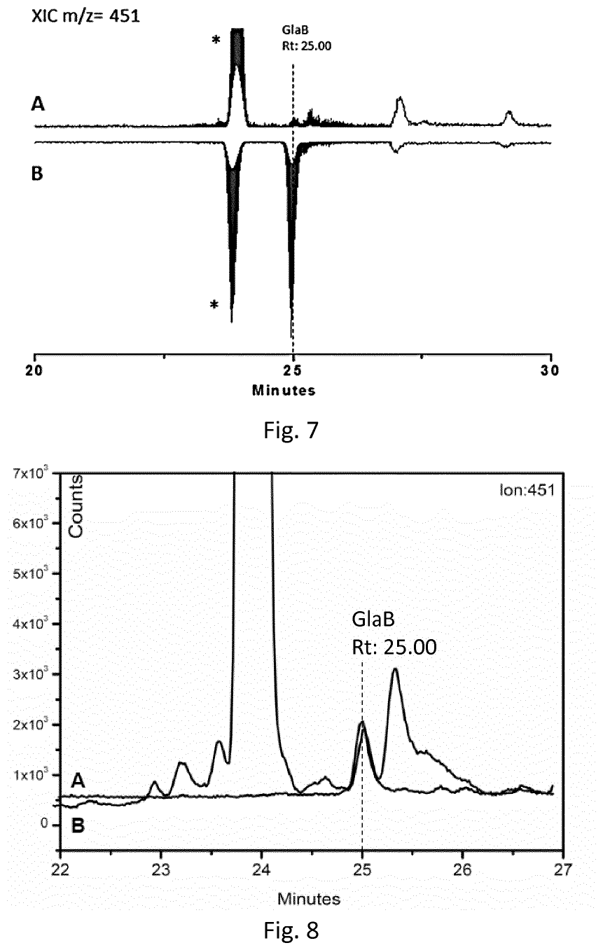

FIG. 7. Extract ion chromatograms (XIC) referred to IN GlaB/PEG-Cholane treated sample (A) and spiked IN sample where a known amount of the GlaB analyte (a spike) was added to the treated brain sample (B). Both traces show the presence of GlaB peak (Rt: 25.00 min). *The peak at Rt: 23.90 min (marked with an asterisk in FIG. 7) is the isotopic abundance with m/z=451 of an unknown peak with m/z=499, present in all brain extracts.

FIG. 8. MS chromatograms acquired in SIM mode of IN GlaB/PEG-Cholane treated mice (A) and of a 0.5 mg/mL standard solution of GlaB (GlaB in MeOH:H₂O 1:1) (B).

### DETAILED DESCRIPTION OF THE INVENTION

### Materials and Methods

### Cell Culture

The GL261 murine glioma cells (Leibniz-Institute DMSZ, ACC802) are grown in an incubator at 37° C. and 5% CO₂ in a culture medium containing D-MEM (GIBCO), 20% fetal bovine serum (GIBCO), antibiotics (100 IU/ml penicillin G, 100 μg/ml streptomycin, 2.5 μg/ml amphotericin B), 2 mM glutamine and 1 mM pyruvate sodium.

### Preparation of GlaB/PEG-Cholane Formulation

GlaB can be synthesised as reported in: Delle Monache, F.; et al. (1977), Gazzetta Chimica Italiana 107(7-8): 403-407 and in WO 2014/207069 A1.

PEG-Cholane can be synthesised as reported in: Salmaso, S. et al. Self-assembling nanocomposites for protein delivery: Supramolecular interactions between PEG-cholane and rh-G-CSF. Journal of Controlled Release 162, 176-184, (2012) and in Ambrosio, E. et al. A novel combined strategy for the physical PEGylation of polypeptides. Journal of Controlled Release 226, 35-46 (2016).

A 1 ml, volume of a GlaB solution (2.0 mg/mL, 4.4 mM) in methanol was added to 1 mL of mPEG_{5 kDa}-cholane solution at different concentrations in methanol. The methanol was removed under reduced pressure and 1 mL of 10 mM phosphate, 0.15 M NaCl, pH 7.4 was added to rehydrate the polymeric film. The mixture was left in a rotary mixer for 48 hours and then centrifuged for 10 min at 14,000 rpm to remove the undissolved GlaB. The supernatant was collected and analyzed to assess GlaB concentration by RP-HPLC.

### Cell Growth Assay

GL261 murine glioma cells were seeded in culture medium in 96-well plates (5000 cells/well). After 4 h they were treated with 5 μM GlaB (C. Ingallina, P. M. Costa, F. Ghirga, R. Klippstein, J. T. Wang, S. Berardozzi, N. Hodgins, P. Infante, S. M. Pollard, B. Botta, K. T. Al-Jamal. Polymeric glabrescione B nanocapsules for passive targeting of Hedgehog-dependent tumor therapy in vitro. Nanomedicine, (2017), 12(7), 711-728. DOI: 10.2217/nnm-2016-0388) or the vehicle in which it is dissolved (DMSO) and every 24 h for three days their growth was evaluated using the dehydrogenation method of MTT salt (3-(4,5-dimetiltiazol-2-yl)-2,5-diphenyltetrazolium bromide, Sigma-Aldrich). The MTT salt was added to each well containing culture medium at the concentration of 0.5 mg/ml, and after 2 h of incubation at 37° C., cell growth was measured by measuring the spectrophotometer, at a wavelength of 570 nm, the absorbance values related to the amount of MTT salt transformed into formazan (insoluble violet-colored salt) simultaneously by live control cells and cells treated with GlaB.

### Procedure a for Tumor Injection, Pharmacological Treatment and Evaluation of Tumor Volume

### Orthotopic Inoculum of Glioma Cells in Mice

Male wild type (C57BL/6) mice were inoculated with murine glioma cells (GL261, Leibniz-Institute DMSZ, ACC802) at the age of 8 weeks (animals weighing an average weight of 22 g). After being anesthetized by intraperitoneal injection of 50 mg/kg Zoletil anaesthetic (combination of tiletamine hypochloride and zolazepam hypochloride) associated with 10 mg/kg Rompun (xylazine), the animals were subjected to orthotopic injection (subcortical/striatum, +1 min anteroposterior, −2 mm lateral to the bregma) of GL261 cells (1×10⁵ cells per mouse) using a stereotaxic apparatus to accurately and reproducibly reach the right striatal region. The cells, resuspended in 5 μl of phosphate buffer, were injected by the use of a Hamilton syringe connected to the stereotaxic apparatus.

### Pharmacological Treatment

GlaB dissolved in the vehicle constituted by ethanol (Sigma-Aldrich #51976): 2-hydroxypropyl-beta-cyclodextrin (Sigma-Aldrich #C0926) solution (10% (w/v) in H₂O) in a 1:5 volume ratio was administered by intraperitoneal injections (ip, 35 mg/kg) and intranasally (in, 4.4 mg/kg), every two days after 7 days from the implantation of tumor cells for a total of 6 days of treatment. Intranasal administration was performed through the so-called “snort delivery”: drops of the medicament were sniffed by the animal, anesthetized with 2.5% Isofluorane, until reaching the upper nasal cavity. This type of administration has also been shown to be effective in humans (13, 14).

### Evaluation of Tumor Volume

The day after the last administration of GlaB, mice were perfused with phosphate buffer and then 4% PFA and their brain isolated, further fixed and frozen. After about 48 h each frozen brain was cut into subsequent coronal slices, 20 μm thick, collected at 100 μm intervals. The determination of tumor volume was performed by histological staining (hematoxylin and eosin) of the brain slices: the tumor mass is recognizable because it has a darker color compared to the remaining healthy tissue. The tumor volume is calculated through analysis of images obtained under a phase contrast microscope (ECLIPSE Ti-S, Nikon) using commercial software released in the public domain (ImageJ, National Institutes of Health of the United States) able to measure the tumor area in each slice, necessary for volume calculation.

### Procedure B for Tumor Injection, Pharmacological Treatment and Evaluation of Tumor Volume

Eight-week-old male C57BL/6 mice were anesthetized with chloralhydrate (400 mg/kg, i.p.) and stereotaxically injected with 1×10⁵ GL261 cells in 5 μl PBS, 2 mm right and 1 mm anterior to the bregma in the striatum at 3 mm depth with a Hamilton syringe (Bonaduz, Switzerland). After 7 days, mice were treated intranasally every two days for six times with GlaB-PEG-Cholane (1.44 mg/Kg, 40 μl) or vehicle PEG-Cholane (40 μl). After 3 weeks from GL261 injection, animals were killed and the brains were isolated. Evaluation of tumor volume was performed as described above in procedure A.

For HPLC analysis, mice were treated as above and sacrificed two hours post one single nasal administration; brains were collected and frozen (−80° C.) until use.

### HPLC

### Sample Preparation

HPLC analyses were performed on brain samples from mice treated with GlaB/PEG-Cholane. The brain sample (500 mg), previously stored at −80° C. for 24 hours, was homogenized with 1 mL of a Phosphate Buffered Saline solution, PBS, using an ultrasound probe. A solution of ZnSO₄ (0.1 M in H₂O, 1 mL) and acetonitrile (1 mL) were added to the mixture, which was further homogenized with ultrasound for 15 minutes. Subsequently, the brain homogenate was stirred for 1 hour at room temperature and was then centrifuged at 3000 g for 5 min at T=4° C. The supernatant was collected. The following procedure to obtain a brain extract was repeated twice. Acetonitrile (1 mL) was added to the brain residue, stirred for 30 minutes at room temperature, then centrifuged at 3000 g for 5 min at T=4° C. and the supernatant collected. Brain extracts samples were centrifuged for five minutes at 5000 rpm and 20 μL of supernatant injected into the HPLC without further treatments.

### Equipment and Chromatographic Conditions

The HPLC chromatographic system used was an UltiMate 3000 RS system (Thermo Fisher Dionex Sunnyvale, Calif.), equipped with an UltiMate 3000 LPG-3400RS Low Pressure Mixing Biocompatible Gradient Pump, an in-line split-loop well plate sampler, a thermostated column ventilated compartment (temperature range: 5-110° C.) and a diode array detector (UltiMate 3000 DAD-3000RS Rapid Separation Diode Array Detector, up to 200 Hz acquisition rate) with a low dispersion 13 μL flow cell.

The stationary phase used was a Titan C18 (100×3.0 mm L×I.D. 1.9 μm).

All chromatographic runs were performed at a flow-rate of 0.4 mL/min with the column equilibrated at 35° C. Solvent A was Water/Acetonitrile 90:10 with 0.1% v/v of formic acid, and solvent B was Acetonitrile/Methanol 50:50 with 0.1% v/v of formic acid. The gradient was set as shown in Table 1.

TABLE 1
Gradient settings for chromatographic runs

| Time (min) | % Solvent B |
|---|---|
| 0 | 20 |
| 3 | 20 |
| 4 | 50 |
| 7 | 50 |
| 37 | 100 |
| 42 | 100 |
| 44 | 20 |
| 48 | 20 |

The LC was directly interfaced to electrospray ionization (ESI) source coupled with a Single Quadrupole-MSQ Plus Detector.

Ion source was operated in positive ESI mode and both Full Scan and SIM (m/z=451, corresponding to the most abundant ion [M+H]⁺ of GlaB, from 22 to 27 minutes) were acquired for each sample. Optimal instrument source parameters for ionization were a cone voltage of 100 V and a Probe Temperature of 550° C.

### Quantitation

Calibration curves were built both in UV (295 nm) and MS in SIM mode, by monitoring the m/z=451 corresponding to the most abundant ion [M+H]⁺ of GlaB.

Calibration standards ranged from 0.128 to 8.2 μg/mL for UV curve (y=0.2574x−0.2042, R2=0.9997) with a Limit of Quantitation, LOQ, (defined for a signal-to-noise ratio >10) of 4.1 ng on column, whereas calibration curve ranged 0.128-8.2 μg/ml for MS (y=11176x−1505.9) showing a correlation coefficient (r²) equal to 0.9991.

A known amount (0.5 μg/mL) of the GlaB analyte (a spike) was added to treated brain sample. The samples and the samples plus spike were then analyzed. The sample with the spike will show a larger analytical response than the original sample due to the additional amount of analyte added to it. The difference in analytical response between the spiked and unspiked samples is due to the amount of analyte in the spike. This provides a calibration point to determine the analyte concentration in the original sample.

### Statistical Analysis

All data are expressed as mean±standard error. All the statistical analysis shown were performed with SigmaPlot 11.0.

### EXAMPLES

### Example 1: Treatment with the Compound of the Invention Reduces In Vitro Glioma Growth

Experimental data in FIG. 1 showed that the GL261 murine glioma cells treated with 5 μM GlaB reduce their growth capacity compared to the same cells treated only with the vehicle (DMSO). The treatment has effect between 24 and 48 h, presumably at the first attempt of tumor cell replication in part dependent on activation of the Hedgehog signalling pathway.

### Example 2: Treatment with the Compound of the Invention Reduces In Vivo Glioma Growth

Experimental data in FIG. 2 showed that intranasal and intraperitoneal administration of GlaB, at the respective concentrations of 4.4 mg/kg and 35 mg/kg, is able to significantly reduce glioma volume in vivo, compared to the volume measured in mice treated with control solution (ethanol:2-hydroxypropyl-beta-cyclodextrin solution, 1:5 v:v) (FIG. 2). Furthermore, intranasal administration is surprisingly effective at a concentration of about 8 times lower than intraperitoneal concentration.

### Example 3: Treatment with a Formulation Comprising the Compound of the Invention and PEG-Cholane Reduces In Vivo Glioma Growth

8-week-old male mice were injected with GL261 cells to obtain an in vivo model of malignant glioma, and treated intranasally with GlaB/PEG-Cholane (1.44 mg/Kg) or vehicle alone (PEG-Cholane) every two days for six times. Treatments started one week after glioma cells injection. The intranasal drug administration, in particular the snort delivery method, was chosen to decrease the concentration of drugs used and possibly to avoid side effects in the other body districts.

As shown in FIG. 3, intranasal administration of PEG-cholane loaded GlaB significantly decreased tumor volume, compared to vehicle treated mice. These data show that PEG-Cholane formulation permitted to obtain the same tumor volume reduction (of about 70%) as that obtained with the formulation in ethanol:2-hydroxypropyl-beta-cyclodextrin solution (1:5 by volume) using a dose of GlaB approximately 3 times lower.

### Example 4: GlaB Brain Level Concentration Measured by HPLC Coupled with Electrospray Mass Spectrometry

GlaB brain level concentration was measured by HPLC coupled with Electrospray Mass Spectrometry. The analytical method was developed in reversed phase and showed a high sensitivity, leading to the quantification of small amounts of GlaB in brain extracts, with a limit of quantitation (LOQ) in MS of 0.26 ng on column. GlaB was quantified in 6 ng on column, thus estimated brain level concentration was 3.6 μg/g of brain as further discussed below (FIG. 4B).

### Example 5: The Compound of the Invention Administered Intranasally Reaches the Brain

Brain extracts of control PEG-Cholane treated mice and intranasally (IN) GlaB/PEG-Cholane treated mice were analyzed and the extract ion chromatograms (XIC) at m/z=451 (most abundant ion [M+H]⁺ of GlaB) were compared.

FIG. 5 shows the presence of GlaB peak in XIC referred to IN treated mice, whereas the same peak was not detected in control sample, as expected. These data demonstrate that GlaB intranasal administration allowed PEG-cholane loaded drug to consistently reach brain district.

### Example 6: Quantification of the Amount of the Compound of the Invention in the Brain

GlaB was quantified in 6 ng on column, thus estimated brain level concentration was 3.6 μg/g of brain. The calibration curve for the determination of GlaB in brain extract shown in FIG. 6 was linear over the range 0.128-8.2 μg/ml. The correlation coefficient (r²) for calibration curve was equal to 0.9991. The equation of calibration curve was y=11176x−1505.9, where y is the peak area and x the concentration of GlaB (μg/ml).

TABLE 2
GlaB concentrations and corresponding area detected
in the calibration curve by HPLC-MS.

| Conc | Area |
|---|---|
| (ug/mL) | (counts*min) |
| 0.128125 | 1272.00 |
| 0.25625 | 2365.00 |
| 0.5125 | 3895.00 |
| 1.025 | 8622.00 |
| 2.05 | 21276.00 |
| 3.075 | 32016.00 |
| 5.125 | 55217.00 |
| 8.2 | 90967.00 |

The identification of GlaB was confirmed by the comparison between IN treated sample and spiked IN sample (FIG. 7), both showing a peak for GlaB with a retention time of 25.00 minutes. This provides a calibration point to determine the GlaB concentration in the original sample.

In order to have an experimental confirmation of GlaB quantitation, IN treated sample was compared to a GlaB standard solution (GlaB in MeOH:H₂O=1:1) whose concentration (5 μg/mL corresponding to 10 ng on column) was in the range of estimated GlaB brain level concentration (6 ng on column). MS traces referred to SIM mode acquisition show a similar area for GlaB peaks in IN sample and in GlaB standard solution, confirming the obtained results (FIG. 8). This experiment provided a further proof of the estimated GlaB brain level concentration.

### REFERENCES

1—Louis D N et al. Acta Neuropathol 2007, 114, 97-109.
2—Stupp et al. The New England Journal of Medicine 2005, 352: 987-996.
3—W. M. Pardridge Neurotherapeutics, 2 (1) (2005), pages 1-2.
4—Kanazawa T et al. Mol Pharm. 2014, 11(5):1471-8.
5—Ohgaki H. et al. Cancer Res 2004, 64, 6892-6899.
6—Jackson M et al. Carcinogenesis. 2015, 36(2):177-85.
7—Pistollato F et al. Stem Cells. 2010, 28(5):851-62.
8—Ulasov I V et al. Mol Med. 2011, 17:103-112.
9—Berman D M et al. Nature 2003, 425: 846-85.
10—Vikas C eta al. PLoS 2015, 10(3): e0116390.
11—Infante P et al. EMBO J. 13 Jan. 2015; 34(2):200-17.
12—Ingallina C et al. Cell Death and Disease 2016; 7(9):e2376.
13—Reger et al. Neurobiol Aging 2006, 451-8.
14—Foltin et al. Biochem Behav 2004, 78(1):93-101.

1. A method of treating of a brain tumor in a patient, comprising administering a compound of formula (1): 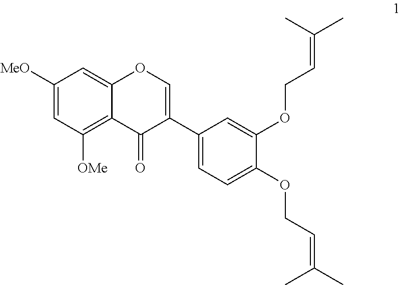 [SMILES: COc1cc(OC)c2c(=O)c(-c3ccc(OCC=C(C)C)c(OCC=C(C)C)c3)coc2c1]
or an analogue thereof or a pharmaceutically acceptable salt thereof intranasally to a patient in need thereof.

2. The method according to claim 1, wherein the brain tumor is a glioma, optionally a glioblastoma.

3. The method according to claim 1, wherein the brain tumor is dependent on the Hedgehog (Hh) signalling pathway.

4. The method according to claim 1, wherein the brain tumor is characterized by the presence of cells that express at least one marker selected from the group consisting of: nestin, Notch3 and the Sonic Hedgehog ligand (SHH).

5. The method according to claim 1, wherein the brain tumor is a primary tumor or a metastasis.

6. The method according to claim 1, wherein the brain tumor is resistant to at least one medicament and/or to radiations.

7. The method according to claim 1, wherein the compound or an analogue thereof or the pharmaceutically acceptable salt thereof is administered every two days, for at least 6 times, optionally at a concentration of from approximately 1 to approximately 10 mg/Kg, and optionally at a concentration of approximately 4.4 mg/Kg or at a concentration of approximately 1.4 mg/Kg.

8. The method according to claim 1, wherein the compound or an analogue thereof or the pharmaceutically acceptable salt thereof is administered in combination with at least one further therapeutic intervention, optionally said further therapeutic intervention being a surgical operation, radiotherapy or a treatment with a further therapeutic agent.

9. (canceled)

10. The method according to claim 1, wherein the compound of formula (1) or an analogue thereof or a pharmaceutically acceptable salt thereof is included in a pharmaceutical composition comprising a pharmaceutically acceptable excipient and/or diluent is selected from the group consisting of: a hydrophilic polymer, a hydrophobic molecule, an alcohol, a cyclodextrin, a polyoxyl hydrogenated castor oil, a polyoxyl castor oil, water and a mixture or conjugate thereof.

11. The method according to claim 10, wherein the hydrophilic polymer is PEG, the hydrophobic molecule is cholane, cholesterol, a phospholipid, or an alkyl chain, the alcohol is ethanol, the cyclodextrin is 2-hydroxypropyl-beta-cyclodextrin, the polyoxyl hydrogenated castor oil is polyoxyl 40 hydrogenated castor oil or polyoxyl 60 hydrogenated castor oil, and/or the polyoxyl castor oil is polyoxyl 35 castor oil.

12. The method according to claim 10, wherein the pharmaceutically acceptable excipient and/or diluent is a conjugate of PEG and Cholane or a mixture of ethanol and 2-hydroxypropyl-beta-cyclodextrin, and the 2-hydroxypropyl-beta-cyclodextrin is optionally in the form of a solution.

13. The method according to claim 12, wherein the PEG and the Cholane are in a 1:1 molar ratio.

14. The method according to claim 12, wherein the 2-hydroxypropyl-beta-cyclodextrin solution is in a concentration of 10% w/v in water.

15. The method according to claim 12, wherein ethanol and the solution are in a 1:5 volume ratio.

16. The method according to claim 1 further comprising administering at least one further therapeutic agent.

17. A pharmaceutical composition comprising a compound of formula (1) or an analogue thereof or a pharmaceutically acceptable salt thereof and a pharmaceutically acceptable excipient and/or diluent, wherein said excipient and/or diluent is selected from the group consisting of: a hydrophilic polymer, a hydrophobic molecule, a polyoxyl hydrogenated castor oil, a polyoxyl castor oil, and a mixture or conjugate thereof.

18. The pharmaceutical composition according to claim 17, wherein the hydrophilic polymer is PEG, the hydrophobic molecule is cholane, cholesterol, a phospholipid, or an alkyl chain, the polyoxyl hydrogenated castor oil is polyoxyl 40 hydrogenated castor oil or polyoxyl 60 hydrogenated castor oil, and/or the polyoxyl castor oil is polyoxyl 35 castor oil.

19. The pharmaceutical composition according to claim 17, wherein the pharmaceutically acceptable excipient and/or diluent is a conjugate of PEG and Cholane.

20. The pharmaceutical composition according to claim 19, wherein the PEG and the Cholane are in a 1:1 molar ratio.

21. The pharmaceutical composition according to claim 17, further comprising at least one further therapeutic agent.
